(19) United States
(12) Patent Application Publication
(10) Pub. No.: US 2026/0096765 A1
(43) Pub. Date: Apr. 9, 2026

(54) Replaceable Overpatch with Embedded Sensors

(71) Applicant: iRhythm Technologies, Inc., San Francisco, CA (US)

(72) Inventor: Steven Robert Keyes, San Francisco, CA (US)

(73) Assignee: iRhythm Technologies, Inc., San Francisco, CA (US)

(21) Appl. No.: 19/341,481

(22) Filed: Sep. 26, 2025

Related U.S. Application Data
(60) Provisional application No. 63/703,628, filed on Oct. 4, 2024.

(51) Int. Cl. A61B 5/282; A61B 5/00; A61B 5/024; A61B 5/1455; A61B 5/257

(52) U.S. Cl. CPC A61B 5/282; A61B 5/257; A61B 5/6833; A61B 5/02416; A61B 5/14552; A61B 2560/0468; A61B 2562/227

(57) ABSTRACT

A replaceable overpatch with embedded sensors is described to attach a main body of a wearable device to a skin surface of a user. For example, the main body houses a processing circuit to process physiological data collected by the embedded sensors of the overpatch. A bottom surface of the main body is configured to face the skin surface during wear and a top surface opposite the bottom surface includes a body connection interface. The overpatch is dimensioned to be placed over the main body to secure the main body to the skin surface and includes an overpatch connection interface that is dimensioned to mate with the body connection interface to form an electrical connection between the overpatch and the main body. In this way, physiological data collected by the embedded sensors can be communicated to the processing circuit without interference caused by proximity to the skin surface.

### RELATED APPLICATIONS

This application claims priority to U.S. Provisional Application No. 63/703,628, titled Replaceable Overpatch with ECG Electrodes, filed Oct. 4, 2024, which is hereby incorporated by reference in its entirety.

### BACKGROUND

Wearable devices for monitoring physiological parameters have become increasingly prevalent in healthcare and consumer applications. These devices typically incorporate various sensors to collect data such as heart rate, electrocardiogram (ECG) signals, and other user health data. However, existing wearable monitors often face challenges in maintaining reliable adhesion to the skin for extended observation periods, particularly in the presence of moisture, movement, and varying environmental conditions. These factors can cause various adverse effects, such as partial or full device detachment, motion artifacts, and signal degradation. Accordingly, the limitations faced by conventional adhesion techniques can offset the advantages provided by wearable devices.

### BRIEF DESCRIPTION OF THE DRAWINGS

FIG. 1 is a block diagram of a non-limiting example of an environment that is operable to employ techniques and components for a replaceable overpatch with embedded sensors as described herein.

FIG. 2 depicts a non-limiting example of a monitoring device.

FIG. 3 depicts a non-limiting example of a main body of a monitoring device that is attachable to an overpatch.

FIG. 4 depicts a non-limiting example of a replaceable overpatch that includes one or more embedded sensors.

FIG. 5 depicts a non-limiting example of attachment of an overpatch to a main body to attach a monitoring device to a skin surface of a user.

FIG. 6 illustrates a non-limiting example in which a main body is compatible with multiple overpatch configurations.

FIGS. 7a and 7b depict non-limiting examples of orientation-dependent functionality of a wearable device.

FIG. 8 depicts a flow diagram as a step-by-step procedure in an example implementation that is performable to attach and operate a wearable device using a replaceable overpatch for physiological monitoring.

### DETAILED DESCRIPTION

Wearable devices offer significant advantages in continuous health monitoring and support real-time tracking of physiological states to provide valuable data for personal wellness and clinical decision-making. However, maintenance of consistent and reliable contact between sensors of the device and a skin surface of a user presents a persistent challenge, particularly during extended wear periods or physical activity. For instance, user movement, failed adhesives, improper device placement, perspiration, or environmental factors can compromise integrity of a sensor-to-skin interface and/or a sensor-to-circuitry interface which can lead to inaccurate readings, data gaps, and unreliable health insights which offset the benefits provided by these devices.

To address these limitations, a wearable device is described that includes a main body which houses a processing circuit and is securable to a skin surface of a user by a replaceable overpatch that includes integrated sensors. For instance, the main body includes a bottom surface that is configured to face the skin surface during wear. The main body further includes a top surface that faces “away from” the skin surface that includes a first portion of a connection interface, e.g., a body connection interface.

The replaceable overpatch is dimensioned to be placed over the main body to secure the main body to the skin surface. For instance, the replaceable overpatch may include a recessed region dimensioned to at least partially surround the main body and an adhesive layer on a skin-facing side configured to adhere to the skin surface around a perimeter of the main body. In some examples, the overpatch can prevent access to one or more ports of the main body, such as to prevent interference during a wear period.

The replaceable overpatch further includes one or more sensors embedded within the overpatch to collect physiological data. For instance, the overpatch includes a flexible substrate with electrocardiogram (ECG) sensors disposed at various locations within the flexible substrate to contact the skin surface and collect cardiac data from the user. The replaceable overpatch can be configured with various types, number, and arrangements of sensors, and thus different overpatches can be selected for particular monitoring scenarios.

The replaceable overpatch further includes conductive traces within the flexible substrate that are electrically coupled to the one or more sensors and can form an electrical connection with a second portion of the connection interface, e.g., an overpatch connection interface. The overpatch connection interface is dimensioned to coincide with (e.g., to mate with) the body connection interface, such as to form an electrical connection between the replaceable overpatch and the main body. Various connection types can be used for the body and overpatch connection interfaces, such as pogo pins, mezzanine connectors, corresponding contact points, and so forth. Thus, physiological data can be collected by the sensors of the replaceable overpatch and communicated to the processing circuit of the main body via the mated connection between the overpatch connection interface and the body connection interface.

Accordingly, this design provides various advantages relative to conventional approaches. By separating the adhesive component (e.g., the removable overpatch) from the main body, the device supports improved reusability of the main body and allows for efficient replacement and customization of the adhesive and sensor elements, This design further supports flexible sensor placement and integration of multiple sensor types. Additionally, embedding sensors directly within the flexible substrate of the overpatch can improve sensor-to-skin contact relative to some conventional approaches by eliminating air gaps that can occur when sensors are mounted on rigid circuit boards or hardware structures. The overpatch can also cover portions of the main body such as to prevent access to service ports during wear, which enhances user safety and reduces a risk of improper use.

Further, the elevated position of the connection interface on the top surface of the main body reduces signal noise due to perspiration by providing a vertical offset between electrical interfaces and the skin surface, which can improve data quality relative to conventional “underpatch” devices with an integrated chassis. Additionally, the devices and techniques described herein support strategic positioning of various sensor types relative to one another, e.g., optical sensors positioned on the bottom surface of the main body at an offset distance from ECG electrodes and/or connection interfaces, such as to reduce interference while maintaining sufficient skin contact. As further described below in more detail, different overpatch designs are operable to activate different sets of sensors and/or processing algorithms, which provides for versatile monitoring capabilities using a consolidated base device.

Accordingly, the techniques, devices, and components described herein address challenges faced by conventional wearable device adhesion approaches to enable reliable, comfortable, and versatile physiological monitoring and support various clinical and consumer applications.

In some aspects, the techniques described herein relate to a wearable device attachable to a skin surface of a user to collect physiological data including: a main body to house a processing circuit to process the physiological data, the main body including a bottom surface configured to face the skin surface during wear of the wearable device and a top surface substantially opposite the bottom surface that includes a body connection interface; and an overpatch dimensioned to be placed over the main body to secure the main body to the skin surface that includes: one or more sensors embedded within the overpatch to collect the physiological data; and an overpatch connection interface dimensioned to mate with the body connection interface to form an electrical connection between the overpatch and the main body and communicate the physiological data to the processing circuit of the main body.

In some aspects, the techniques described herein relate to a wearable device, wherein the one or more sensors include electrocardiogram (ECG) electrodes, and the overpatch includes conductive traces embedded within a flexible substrate that are electrically coupled to the ECG electrodes and to the overpatch connection interface.

In some aspects, the techniques described herein relate to a wearable device, wherein the main body includes one or more curved edges between the top surface and one or more side surfaces of the main body, the one or more curved edges having a curve radius to accommodate stress parameters of the conductive traces to reduce mechanical stress on the conductive traces.

In some aspects, the techniques described herein relate to a wearable device, wherein the overpatch includes a recessed region dimensioned to at least partially surround the main body and an adhesive layer on a skin-facing side of the overpatch configured to adhere to the skin surface of the user around a perimeter of the main body.

In some aspects, the techniques described herein relate to a wearable device, wherein the body connection interface includes one or more pogo pins, mezzanine connectors, or insertable zip connectors, and the overpatch connection interface includes corresponding connectors configured to mate with the body connection interface.

In some aspects, the techniques described herein relate to a wearable device, wherein the main body is dimensioned to position a mated connection between the body connection interface and the overpatch connection interface at a vertical offset from the skin surface at a height of the main body to reduce incidence of signal noise due to perspiration.

In some aspects, the techniques described herein relate to a wearable device, wherein the bottom surface of the main body further includes one or more optical sensors to contact the skin surface to collect optical data and the overpatch is dimensioned to position the one or more sensors at an offset distance from the one or more optical sensors, the offset distance configured to reduce noise between the one or more sensors and the one or more optical sensors.

In some aspects, the techniques described herein relate to a wearable device, wherein the overpatch is configured to prevent access to one or more service ports of the main body during wear of the wearable device.

In some aspects, the techniques described herein relate to a wearable device, wherein the main body further includes one or more mechanical interface components, and the overpatch includes one or more corresponding mechanical interface components configured to engage with the one or more mechanical interface components of the main body to further secure the overpatch to the main body.

In some aspects, the techniques described herein relate to a replaceable overpatch to attach a main body of a wearable monitoring device to a user, the replaceable overpatch including: a flexible substrate dimensioned to be placed over the main body of the wearable monitoring device, the flexible substrate including a sensing region to contact a skin surface of the user and a coupling region to attach the replaceable overpatch to the main body; one or more sensors embedded within the sensing region to collect physiological data from the user; one or more conductive traces embedded within the flexible substrate electrically coupled to the one or more sensors; and an overpatch connection interface electrically coupled to the one or more conductive traces configured to mate with a corresponding body connection interface of the main body to enable transmission of the physiological data to a processing circuit of the main body.

In some aspects, the techniques described herein relate to a replaceable overpatch, wherein the one or more sensors include electrocardiogram (ECG) electrodes embedded within the flexible substrate.

In some aspects, the techniques described herein relate to a replaceable overpatch, wherein the sensing region includes an adhesive layer on a skin-facing side of the flexible substrate configured to adhere to the skin surface of the user and the coupling region includes a recessed region dimensioned to at least partially surround the main body of the wearable monitoring device.

In some aspects, the techniques described herein relate to a replaceable overpatch, wherein the flexible substrate is configured to prevent access to one or more service ports of the main body when the replaceable overpatch is attached to the wearable monitoring device.

In some aspects, the techniques described herein relate to a replaceable overpatch, further including one or more mechanical interface components configured to engage with corresponding mechanical interface components on the main body to secure the replaceable overpatch to the main body.

In some aspects, the techniques described herein relate to a replaceable overpatch, wherein the overpatch connection interface includes one or more conductive pads or flexible circuit board terminals configured to mate with corresponding connectors of the body connection interface.

In some aspects, the techniques described herein relate to a replaceable overpatch, further including an additional overpatch connection interface, and wherein connection of the overpatch connection interface with the body connection interface activates a first set of sensors and a first processing algorithm and connection of the additional overpatch connection interface with the body connection interface activates a second set of sensors and a second processing algorithm.

In some aspects, the techniques described herein relate to a replaceable overpatch, wherein the flexible substrate includes at least one of a protective layer, a moisture-wicking layer, a conductive layer, an insulating layer, a breathable layer, a stretchable layer, or an antimicrobial layer.

In some aspects, the techniques described herein relate to a main body of a wearable device attachable to a skin surface of a user by a replaceable overpatch to collect physiological data from the user, the main body including: a housing that includes a processing circuit to process the physiological data, a bottom surface configured to face the skin surface during wear of the wearable device, and a top surface substantially opposite the bottom surface; and a body connection interface disposed on the top surface of the housing configured to form an electrical connection with a corresponding overpatch connection interface of the replaceable overpatch to receive the physiological data from one or more sensors of the replaceable overpatch.

In some aspects, the techniques described herein relate to a main body, wherein the housing is dimensioned to position a mated connection between the body connection interface and the overpatch connection interface at a vertical offset from the skin surface at a height of the housing to reduce incidence of signal noise due to perspiration.

In some aspects, the techniques described herein relate to a main body, further including an additional body connection interface, and wherein responsive to connection of the body connection interface with the overpatch connection interface the processing circuit activates a first set of sensors of the replaceable overpatch and a first processing algorithm and responsive to connection of the additional body connection interface with the overpatch connection interface the processing circuit activates a second set of sensors of the replaceable overpatch and a second processing algorithm.

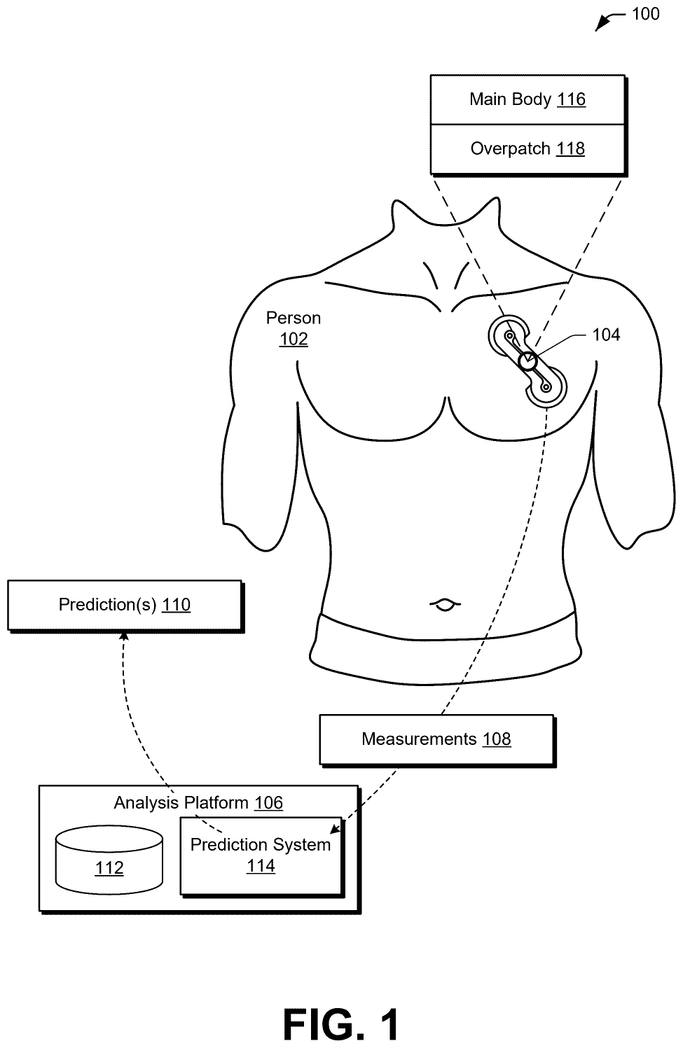

FIG. 1 is a block diagram of a non-limiting example 100 of an environment that is operable to employ techniques and components for replaceable overpatch with embedded sensors as described herein. The illustrated example 100 includes person 102, who is depicted wearing a monitoring device 104. The illustrated environment also includes an analysis platform 106. The analysis platform 106 may be connected to the monitoring device 104 via one or more wireless connections directly or via one or more wired and/or wireless connections and one or more intermediate devices, such as a computing device associated with the person 102, network routing devices and equipment, server devices, and/or the Internet, to name just a few.

The monitoring device 104 may be utilized to monitor one or more aspects of the person 102, such as to generate measurements 108. In some scenarios, for instance, the monitoring device 104 may be provided to record electrical activity of the person 102's heart over an observation period, e.g., lasting some number of seconds or minutes, lasting multiple days, and so on. By way of example, the person 102 may have a magnitude of his or her heart's electrical potential monitored over time to produce one or more electrocardiograms, which may be used to predict any of a variety of events. Alternatively or in addition, the monitoring device 104 may be used to output measurements 108 (e.g., a time sequence of measurements such as a time sequence of electric potential measurements), which may indicate an observation or be used to generate a prediction of one or more events.

In connection with the monitoring device, instructions may be provided to the person 102 that instruct the person 102 how to operate the monitoring device 104 and/or how to behave (e.g., sleep, perform activity) while wearing monitoring device 104. In one or more implementations, the instructions may be provided as part of a kit, e.g., written instructions. Alternately or additionally, the analysis platform 106 may cause the instructions to be communicated to and output (e.g., for display and/or audio output) via a computing device associated with the person 102. In one or more implementations, the analysis platform 106 may wait to provide these instructions for output after a predetermined amount of time of an observation period has lapsed (e.g., two days) while wearing the monitoring device 104 and/or based on patterns in the aspects of the person 102 being measured.

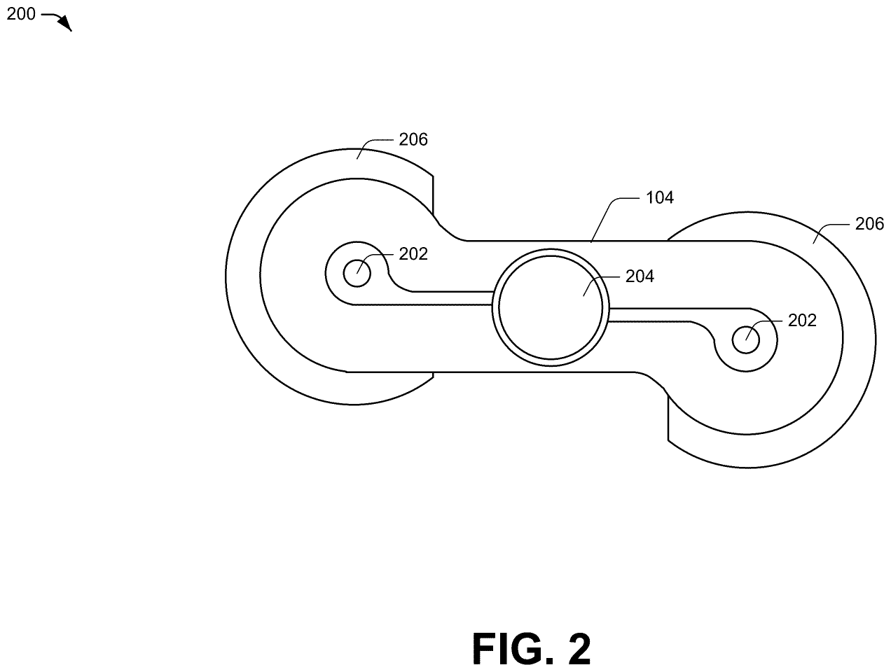

The monitoring device 104 may be configured in a variety of ways to monitor one or more aspects of the person 102. Moreover, the form factor depicted in FIGS. 1 and 2 is just one example form factor, and the form factor of the monitoring device 104 may differ in variations. It is to be appreciated that the monitoring device 104 may be configured with one or more sensors, examples of which include one or more of: a plurality of electrodes (e.g., that can be placed on the skin of the person), an accelerometer, and a pulse oximeter (e.g., to measure and record oxygen saturation (SpO2) and/or produce a photoplethysmogram of the person 102), to name just a few. Certainly, the monitoring device 104 may be configured with any of a variety of types of sensors without departing from the described techniques.

Although the monitoring device 104 may be configured in a similar manner as monitoring devices used for clinically monitoring patients, in one or more implementations, the monitoring device 104 may be configured differently than the devices used for monitoring and/or diagnosing patients clinically. By way of example, and not limitation, the monitoring device 104 may be configured as a ring, a watch, a patch, and/or a strap, to name just a few form factors. Alternatively or additionally, the monitoring device 104 may have a similar form factor as for clinical settings, but have different functionality, such as functionality that prevents a wearer from viewing the measurements 108.

In one or more implementations, the monitoring device 104 may be configured to offload measurements 108 during the course of the observation period. By way of example, the monitoring device 104 may offload the measurements 108 by transmitting them via a wired or wireless connection to an external computing device, e.g., at predetermined time intervals and/or responsive to establishing or reestablishing a connection with the computing device. In one or more implementations, the measurements 108 and/or other data from the monitoring device 104 may be compressed by the monitoring device 104 for wireless transmission, e.g., using one or more of a variety of data compression techniques. Compression of the sensor data in this way can reduce battery usage of the monitoring device 104 during the observation period and facilitate wear during assessments of physiological conditions.

To the extent that the monitoring device 104 may be configured to store the measurements 108 for an entirety of an observation period, in one or more implementations, the monitoring device 104 may be configured without wireless transmission means, e.g., without any antennae to transmit the measurements 108 wirelessly and without hardware or firmware to generate packets for such wireless transmission. Instead, the monitoring device 104 may be configured with hardware to communicate the measurements 108 via a physical, wired coupling. In such scenarios, the monitoring device 104 may be “plugged in” to extract the measurements 108 from the device's storage.

Accordingly, the monitoring device 104 may be configured with one or more ports to enable wired transmission of the measurements 108 to an external computing device. Examples of such physical couplings may include micro universal serial bus (USB) connections, mini-USB connections, and USB-C connections, to name just a few. Although the monitoring device 104 may be configured for extraction of the measurements 108 via wired connections as discussed just above, in different scenarios, the monitoring device 104 may alternately or additionally be configured to offload the measurements 108 over one or more wireless connections.

Once the monitoring device 104 produces the measurements 108, the measurements 108 are provided to the analysis platform 106. As noted above, the measurements 108 may be communicated to the analysis platform 106 over wired and/or wireless connection(s).

In scenarios where the analysis platform 106 is implemented partially or entirely on the monitoring device 104, for instance, the measurements 108 may be transferred over a bus from the device's local storage to a processing system of the device. In scenarios where the monitoring device 104 is configured to generate one or more predictions 110 by processing the measurements 108, the monitoring device 104 may also be configured to provide the generated one or more predictions 110 as output, e.g., by communicating the one or more predictions 110 to an external computing device. In other scenarios, the measurements 108 may be processed by an external computing device configured generate one or more predictions 110. For example, the measurements 108 (and/or other measurements such as accelerometer data and oxygen saturation (SpO2) measurements) may be processed by a smartphone associated with the user, a smartphone or other dedicated device associated with the monitoring device 104, and/or one or more server computers at a data center or other location that can be utilized by an entity associated with the monitoring device 104, to name just a few. In other words, those other devices may implement at least a portion of the analysis platform 106 and/or a prediction system 114.

In one or more implementations, the monitoring device 104 is configured to transmit the measurements 108 to an external device over a wired connection with the external device, e.g., via USB-C or some other physical, communicative coupling. Here, a connector may be plugged into the monitoring device 104 or the monitoring device 104 may be inserted into an apparatus having a receptacle that interfaces with corresponding contacts of the device. The measurements 108 may then be obtained from storage of the monitoring device 104 via this wired connection, e.g., transferred over the wired connection to the external device. Such a connection may be used in scenarios where the monitoring device 104 is mailed by the person 102 after the observation period, such as to a health care provider, telemedicine service, provider of the monitoring device 104, or medical testing laboratory.

Alternatively or additionally, the monitoring device 104 may provide the measurements 108 to the analysis platform 106 by communicating the measurements 108 over one or more wireless connections. For example, the monitoring device 104 may wirelessly communicate the measurements 108 to external computing devices, such as a mobile phone, tablet device, laptop, smart watch, other wearable health tracker, and so on. Accordingly, the monitoring device 104 may be configured to communicate with external devices using one or more wireless communication protocols or techniques. By way of example, the monitoring device 104 may communicate with external devices using one or more of Bluetooth (e.g., Bluetooth Low Energy links), near-field communication (NFC), Long Term Evolution (LTE) standards such as 5G, and so forth. Monitoring devices 104 may be configured with corresponding antennae and other wireless transmission means in scenarios where the measurements 108 are communicated to an external device for processing. In those scenarios, the measurements 108 may be communicated to the analysis platform 106 in various manners, such as at predetermined time intervals (e.g., every day, every hour, or every five minutes), responsive to occurrence of some event (e.g., filling a storage buffer of the monitoring device 104), or responsive to an end of an observation period, to name just a few.

Thus, regardless of where the analysis platform 106 is implemented (e.g., at the monitoring device 104, at a smartphone associated with the person 102, or at a server device), the analysis platform 106 obtains the measurements 108 produced by the monitoring device 104. In one or more implementations, the analysis platform 106 also obtains other measurements produced by the monitoring device 104 and/or any other devices used during the observation period, e.g., a smartwatch, chest strap, etc. As noted above, examples of such additional measurements include but are not limited to accelerometer data and/or oxygen saturation (SpO2) measurements.

In one or more implementations, the analysis platform 106 may be implemented in whole or in part at the monitoring device 104. Alternately or additionally, the analysis platform 106 may be implemented in whole or in part using one or more computing devices external to the monitoring device 104, such as one or more computing devices associated with the person 102 (e.g., a mobile phone, tablet device, laptop, desktop, or smart watch) or one or more computing devices associated with a service provider (e.g., a health care provider, a telemedicine service, a service corresponding to the provider of the monitoring device 104, a medical testing laboratory service, and so forth). In the latter scenario, the analysis platform 106 may be implemented at least in part on one or more server devices.

In the illustrated example 100, the analysis platform includes storage device 112. In accordance with the described techniques, the storage device 112 is configured to maintain the measurements 108 and/or other measurements or information processed by the prediction system 114 to generate one or more predictions 110. The storage device 112 may represent one or more databases and also other types of storage capable of storing the measurements 108 and/or other types of measurements. The storage device 112 may also store a variety of other data, such as personal information, demographic information describing the person 102, information about a health care provider, information about an insurance provider, payment information, prescription information, determined health indicators, account information (e.g., username and password), and so forth. The storage device 112 may also maintain data of other users of a user population.

In the illustrated example 100, the analysis platform 106 also includes the prediction system 114. The prediction system 114 represents functionality to process the measurements 108 to generate the one or more prediction(s) 110. Alternatively or in addition, the prediction system 114 may output one or more time sequences indicating an observation or prediction of one or more events, over time. It is also to be appreciated that the prediction system 114 may output different combinations of multiple predictions in variations.

In at least one implementation, the prediction system 114 uses machine learning to generate one or more predictions 110. By way of example and not limitation, the prediction system 114 may include one or more neural networks trained based on the historical measurements and the historical outcome data of a user population. The prediction system 114 may include one or multiple machine learning models (e.g., an ensemble of models). Alternatively or additionally, the prediction system 114 may include logic (a machine learning model and/or other types of logic) to pre-process the obtained measurements, such as to extract various cardiovascular and/or other features from the sequences of measurements. The illustrated example 100 also includes prediction(s) 110, which corresponds to the output of the prediction system 114.

In the illustrated example, the monitoring device 104 is depicted to include a main body 116 and an overpatch 118. As further described in more detail below, the main body 116 houses a processing circuit to process physiological data (e.g., measurements 108) that are collected by one or more sensors embedded with the overpatch 118. The overpatch 118, for instance, is configured to secure the main body 116 to a skin surface of the person 102 and further forms an electrical connection with the main body 116 through a connection interface positioned on a top surface of the main body 116 to enable sensor data transmission while maintaining a vertical offset from the skin surface such as to reduce biological noise. The overpatch 118 may be replaced independently of the main body 116, such as to allow for customizable sensor configurations and improved device reusability while providing reliable adhesion and physiological monitoring capabilities.

FIG. 2 depicts a non-limiting example 200 of a monitoring device. The illustrated example 200 depicts the monitoring device 104.

In accordance with the described techniques, the monitoring device 104 includes one or more sensors 202, examples of which include but are not limited to one or more pairs of electrodes, an accelerometer, a pulse oximeter, and sweat sensors, to name just a few. The monitoring device 104 may also include a transmitter 204. In this example 200, the monitoring device 104 further includes one or more adhesive portions 206. In operation, the monitoring device 104 is configured to be applied to the skin via the one or more adhesive portions 206, such that, for example, the one or more sensors 202 are positioned to detect and record the electrical activity of the person 102's heart, e.g., to produce an electrocardiogram (ECG and/or EKG). In at least one implementation, the monitoring device 104 may be removed by peeling the one or more adhesive portions 206 off of the skin.

It is to be appreciated that the monitoring device 104 and its various components are simply one form factor, and the monitoring device 104 and its components may have different form factors without departing from the spirit or scope of the described techniques.

In one or more implementations, the monitoring device 104 may include a processor and/or memory (not shown). The monitoring device 104, by leveraging the processor, may generate the measurements 108 based on the communications with one or more sensors 202 that are indicative of some aspect of the person 102, such as the person 102's heart's electrical activity. In one or more implementations, the processor further generates one or more communicable packages of data that include one or more of the measurements 108 and/or other measurements, such as accelerometer data and oxygen saturation (SpO2) measurements. Alternately or additionally, the processor produces and/or causes storage of other data, which may be used for predicting classifications of physiological conditions, e.g., sleep apnea.

In implementations where the monitoring device 104 is configured for wireless transmission, the transmitter 204 may transmit the measurements 108 wirelessly as a stream of data to a computing device. In one or more implementations, for instance, the monitoring device 104 is configured to transfer (e.g., transmit and/or receive) information (e.g., electrical potential measurements) via a Bluetooth Low Energy (BLE) connection. Alternately or additionally, the monitoring device 104 may buffer the measurements 108 (e.g., in memory) and cause the transmitter 204 to transmit the buffered measurements later at various intervals, e.g., time intervals (every second, every thirty seconds, every minute, every five minutes, every hour, and so on), storage intervals (when the buffered measurements reach a threshold amount of data), and so forth.

### Replaceable Overpatch with Embedded Sensors

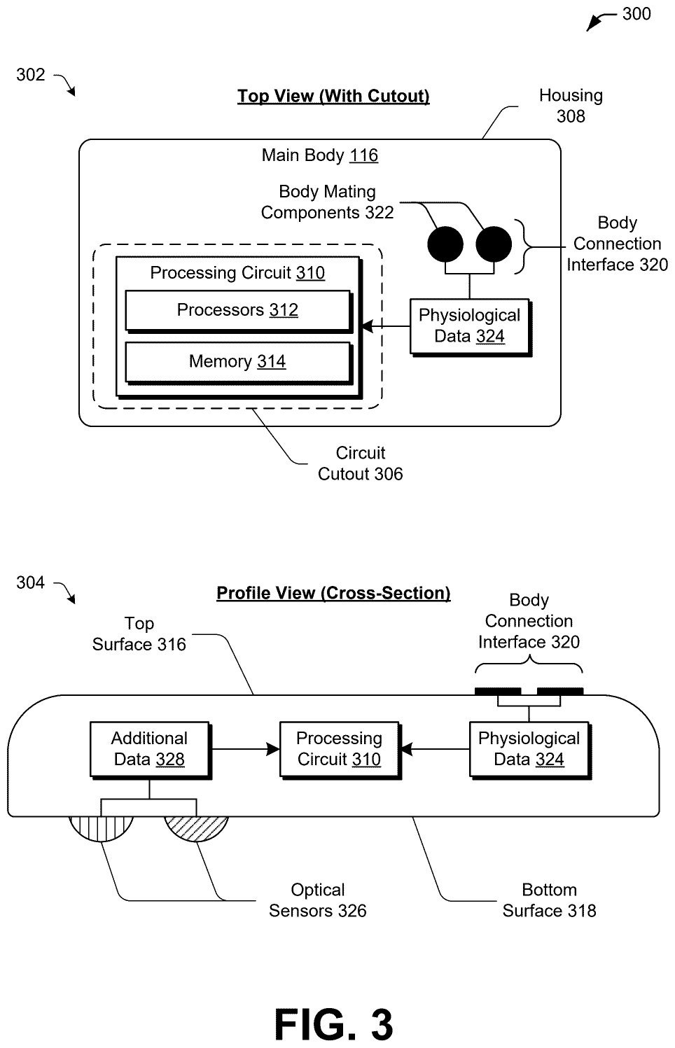

FIG. 3 depicts a non-limiting example 300 of a main body 116 of a monitoring device 104 that is attachable to an overpatch 118. FIG. 3 includes a top view 302 and a profile view 304 of the main body 116.

The top view 302 depicts the main body 116 from above and further illustrates a circuit cutout 306 in a housing 308 of the main body 116, such as to depict internal components of the main body 116. For instance, the circuit cutout 306 depicts a processing circuit 310, which includes processors 312 and memory 314, that is disposed within the housing 308 of the main body 116.

The housing 308 includes a top surface 316 and a bottom surface 318. The top surface 316 includes a body connection interface 320, which includes body mating components 322. The body connection interface 320 is configured to interact with one or more external components. For instance, the body connection interface 320 can mate with a corresponding interface of the overpatch 118 and receive data, e.g., physiological data 324, from one or more sensors as described in more detail below. In this example, the bottom surface 318 further includes optical sensors 326, which can be configured to collect additional data 328 for processing by the processing circuit 310.

In various embodiments, the housing 308 is configured as a sealed enclosure such as to protect internal components (e.g., the processing circuit 310) during a wear period and/or to support cleaning of the main body 116 between uses. For instance, the sealed configuration allows the main body 116 to be reused with multiple replaceable overpatches 118 while maintaining integrity of internal components.

The housing 308 may be constructed from a variety of materials, with various shapes, configurations, and sizes such as to accommodate different monitoring scenarios. In some implementations, the housing 308 may be formed from and/or include biocompatible materials such as medical-grade plastics, silicones, or polymers that are safe for extended skin contact. The housing 308 may include antimicrobial materials such as to reduce infection risk during prolonged wear periods, e.g., 1-14 days.

In various implementations, the housing 308 may be configured with different geometric profiles, such as rectangular, circular, oval, or ergonomic contours that conform to body anatomy. The housing 308 may include textured surfaces, smooth finishes, or grip-enhancing features such as to facilitate handling during assembly and removal. The housing 308 can incorporate one or more flexible sections and/or rigid segments such as based on an intended application.

The housing 308 may be dimensioned in various sizes to accommodate different patient populations, such as pediatric, adult, or specialized form factors for particular body locations. In some implementations, the housing 308 may include color-coding, visual indicators, and/or identification markings such as to distinguish between different device types or configurations. The housing 308 may further include environmental protection features such as dust resistance, moisture barriers, or temperature tolerance specifications to maintain functionality across various. In at least one example, the housing 308 accommodates one or more service ports (e.g., USB, mini-USB, USB-C, charging ports, etc.) which may be covered by the overpatch 118 during wear as further described in more detail below.

In at least one example, the housing 308 includes one or more mechanical interfaces, such as to facilitate secure attachment to the overpatch 118. By way of example and not limitation, a mechanical interface may include one or more slots, tabs, grooves, ridges, snap-fit components, or alignment pins that correspond with complementary features on the overpatch 118. The one or more mechanical interfaces can be positioned at various locations around the perimeter of the housing 308 and/or distributed across the top surface 316 such as to provide various attachment points. In some implementations, the mechanical interfaces may be designed to provide tactile or audible feedback when proper connection is achieved, such as to confirm secure attachment of the main body 116 to the overpatch 118.

In this example, the top surface 316 of the housing 308 is substantially opposite the bottom surface 318 and is configured to face “away from” the skin surface during wear while the bottom surface 318 of the housing 308 is configured to face the skin surface during wear. In the illustrated example, the main body 116 is depicted to include optical sensors 326 disposed on the bottom surface 318. The optical sensors 326, for instance, may include photoplethysmogram (PPG) sensors or other optical measurement devices. In some implementations, the optical sensors 326 collect the additional data 328 such as heart rate, blood oxygen levels, or other physiological parameters through optical measurement techniques. The top surface 316 may further include additional components and/sensors, e.g., one or more ambient light sensors.

The processing circuit 310 is configurable to store and/or process the physiological data 324 (and/or the additional data 328) received from sensors of a replaceable overpatch 118, e.g., as transmitted through the body connection interface 320. In some examples, the processors 312 execute one or more algorithms and/or leverage one or more machine learning models stored in the memory 314 to analyze the physiological data 324. The memory 314 may store various data such as but not limited to processing algorithms, calibration data, and/or temporary measurement data during operation of the main body 116. In various embodiments, the processing circuit 310 may perform one or more actions of and/or leverage functionality of the analysis platform 106 and/or the prediction system 114.

The body connection interface 320 may include various types of connectors including various body mating components 322, such as to establish electrical communication with the overpatch 118. By way of example and not limitation, the body mating components 322 include one or more pogo pins, mezzanine connectors, spring-loaded contacts, magnetic connectors, conductive pads/apertures, flexible circuit board terminals, insertable zip connectors, conductive elastomer interfaces, or other electrical connection mechanisms. As further described below, the body connection interface 320 can be positioned on the top surface 316 such as to maintain separation from a skin surface and thus reduce exposure to biological noise due to moisture or perspiration.

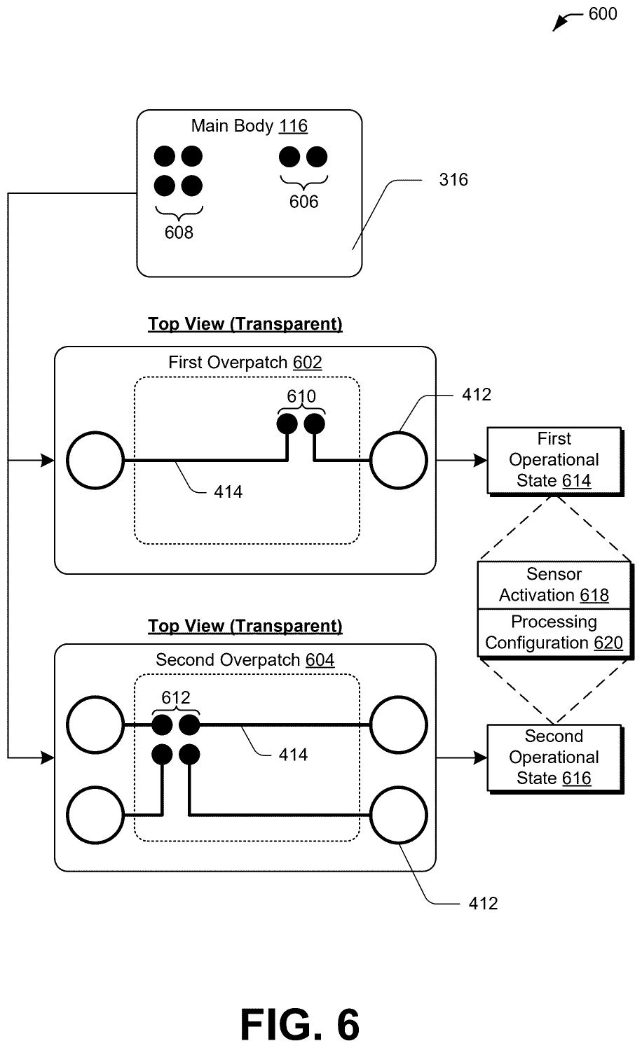

The main body 116 may further include a plurality of body connection interfaces 320, such as to enable connection with different types of overpatches, support different functionality based on which body connection interface 320 is activated, and/or to support different orientations of the same overpatch. For instance, as further described below with reference to FIG. 6, the main body 116 may include multiple connection interfaces positioned at different locations to accommodate various overpatch configurations and/or styles. In this way, the main body 116 is operable to activate various sensor combinations and/or processing algorithms based on which connection interface is engaged, thereby enhancing versatility of the monitoring device 104 for different monitoring scenarios.

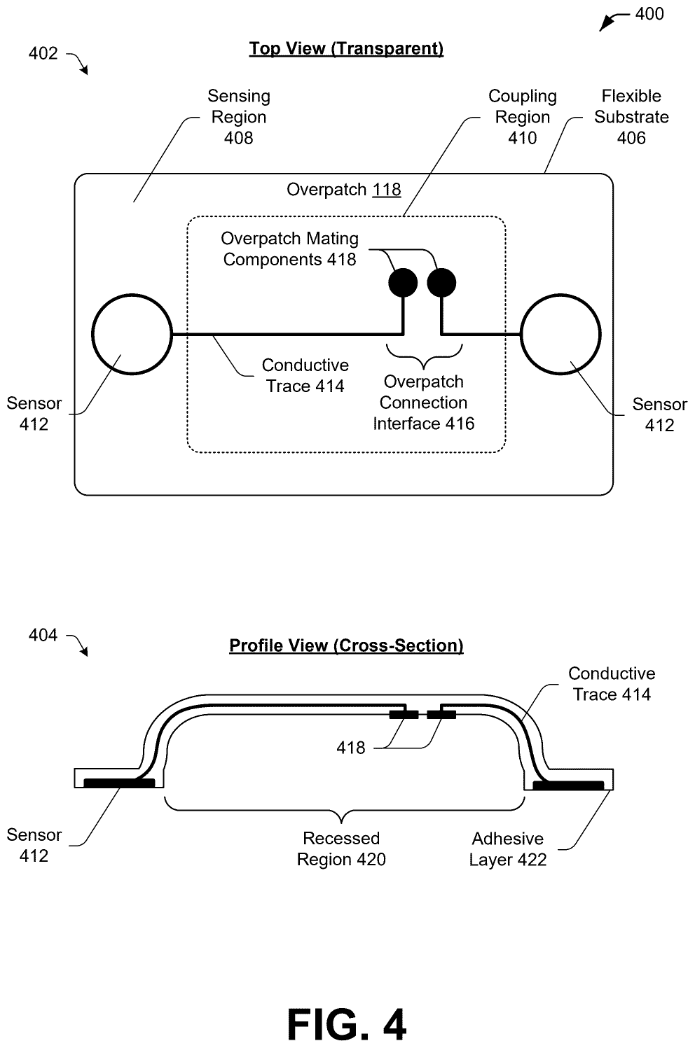

FIG. 4 depicts a non-limiting example 400 of a replaceable overpatch 118 that includes one or more embedded sensors in a top view 402 and a profile view 404.

The top view 402, for instance, depicts a transparent view of the overpatch 118 such that one or more components that may be disposed within the overpatch 118 and/or positioned on a skin-facing side of the overpatch 118 are visually depicted. That is, in various examples the components depicted in the top view 402 are not necessarily disposed on a top surface of the overpatch 118. The profile view 404 depicts a cross-section of the overpatch 118, such as to depict various internal components and spatial relationships of the components.

In this example 400, the overpatch 118 is configured to attach to the main body 116, e.g., the main body 116 as described above with respect to FIG. 3. The overpatch 118 is further configured to secure the main body 116 to a skin surface of a person 102. As part of the attachment, the overpatch 118 includes various components that are operable to form a mechanical and/or an electrical connection with the main body 116 such as to transmit physiological data 324 to the processing circuit 310.

As illustrated in the top view 402, the overpatch 118 includes a flexible substrate 406. The flexible substrate 406, for instance, forms a base structure that supports and integrates various components while maintaining flexibility to conform to surface contours during use. The flexible substrate 406 may be dimensioned to be placed over the main body 116 of the monitoring device 104 such as to enable secure attachment and support accurate physiological data collection. The flexible substrate 406 can include a variety of suitable materials, such as polyethylene terephthalate (PET), polyimide, polyurethane, silicone, thermoplastic elastomers, biocompatible polymers, and so forth. The flexible substrate 406 may further be formed in a variety of shapes, such as but not limited to rectangular, circular, oval, elliptical, kidney-shaped, butterfly-shaped, hourglass-shaped, figure-eight configurations, irregular contours to conform to particular body regions, elongated strips, square profiles, hexagonal arrangements, or custom geometries tailored to particular monitoring applications.

In various examples, the flexible substrate 406 includes one or more layers to provide various functions. For instance, the flexible substrate 406 can include at least one of a protective layer, a moisture-wicking layer, a conductive layer, an insulating layer, a breathable layer, a stretchable layer, or an antimicrobial layer. A protective layer, for instance, may shield internal components from environmental factors. A moisture-wicking layer may be configured to support user comfort and reduce skin irritation during extended wear. The conductive layer can include various conductive elements and/or support electrical transmission, such as to support functionality of one or more elements embedded within the flexible substrate 406. An insulating layer is configurable to separate conductive elements such as to prevent unwanted electrical connections. A breathable layer may allow air circulation in a controlled manner, such as to improve user comfort and augment wear time of the monitoring device 104. A stretchable layer may accommodate body movement during wear, and an antimicrobial layer may include various elements operable to reduce risk of infection during extended wear periods.

The overpatch 118 is further illustrated to include a sensing region 408 and a coupling region 410. The sensing region 408, for instance, is region of the flexible substrate 406 that is configured to contact a skin surface of a user, such as to attach the monitoring device 104 to the user. The coupling region 410, for instance, represents one or more portions of the flexible substrate 406 configured to facilitate mechanical and/or electrical attachment of the overpatch 118 to the main body 116 of the monitoring device 104.

The sensing region 408 includes one or more sensors 412 embedded within the flexible substrate 406 which are configurable to collect a variety of physiological data 324 from a user. In at least one example, the sensors 412 include one or more leads of an electrocardiogram (ECG). This is by way of example and not limitation, and various sensor types and configurations are considered.

For instance, the sensors 412 may include one or more electrocardiogram (ECG) electrodes, electromyogram (EMG) sensors, galvanic skin response (GSR) sensors, temperature sensors, impedance sensors, strain gauges, pressure sensors, pH sensors, sweat analysis sensors, bioimpedance sensors, glucose sensors, or accelerometers. The sensors 412 may be positioned at various locations within the sensing region 408 such as to optimize contact with the skin surface and data collection quality. In some cases, the sensors 412 may include silver/silver chloride electrodes embedded in hydrogel materials to facilitate electrical contact with skin. The sensors 412 may be configured in different arrangements, such as linear arrays, circular patterns, or custom layouts based on an intended monitoring application.

In some examples, the sensor 412 include different sets of sensors that are activatable in various combinations. For instance, the overpatch 118 may incorporate multiple sensor types such as ECG electrodes, temperature sensors, and bioimpedance sensors that can be selectively activated based on monitoring requirements. As further described in more detail below, activation patterns of the sets of sensors may be based on which of a plurality of connection interfaces is engaged with the main body 116, based on software configurations within the processing circuit 310, and/or based on a physical orientation of the overpatch 118 relative to the main body 116.

The sensors 412 can be electrically connected via one or more conductive traces 414. For instance, one or more conductive traces 414 may be embedded within the flexible substrate 406 and electrically coupled to the sensor 412. The conductive trace 414 can provide an electrical pathway between the sensor 412 and other components of the overpatch 118. In some implementations, the conductive trace 414 includes silver ink traces printed on the flexible substrate 406, however various suitable materials are considered. The conductive trace 414 may extend across portions of the flexible substrate 406 to connect sensors 412 to various interface components, such as to communicate physiological data 324 collected by the one or more sensors 412.

For instance, the coupling region 410 is depicted to include an overpatch connection interface 416, which includes one or more overpatch mating components 418. The overpatch connection interface 416 may be electrically coupled to the conductive traces 414 thus providing an electrical connection between the sensors 412 and the overpatch connection interface 416. The overpatch connection interface 416 can be configured to mate with a corresponding body connection interface 320 of the main body 116, such as via engagement of the overpatch mating components 418 with the body mating components 322. Accordingly, the overpatch connection interface 416 enables transmission of physiological data 324 collected by the sensors 412 to a processing circuit 310 of the main body 116.

A variety of properties (e.g., styles, configurations, number, etc.) of the overpatch connection interface 416 and the overpatch mating components 418 are considered. For instance, the overpatch connection interface 416 may include various connector types such as conductive pads, flexible circuit board terminals, spring-loaded contacts, magnetic connectors, or insertable zip connectors that correspond to body mating components 322 of the body connection interface 320. The overpatch connection interface 416 and/or the body connection interface 320 can include one or more male/female connection components. A number of overpatch mating components 418 may vary based on complexity of a particular monitoring application, such as a simple two-point connection for a basic ECG monitoring or a multi-point array for an advanced physiological monitoring scenario with multiple sensor types.

The overpatch 118 may further include more than one overpatch connection interface 416, such as to support various functionality of the monitoring device 104. For example, different overpatch connection interfaces 416 can correspond to different sets of sensors 412 and/or different functionality to be performed by the sensors 412. The multiple overpatch connection interfaces 416 may be arranged in a variety of patterns, such as linear arrays, circular arrangements, or custom configurations based on particular monitoring scenarios. In some implementations, the overpatch connection interface 416 may include redundant connection points to ensure reliable data transmission in examples in which one or more connections are compromised during wear.

In some examples, the coupling region 410 is dimensioned to at least partially enclose the main body 116. That is, a geometry of the coupling region 410 can correspond to a geometry (e.g., shape, size, etc.) of the housing 308 of the main body 116. Accordingly, the coupling region 410 may include one or more contoured surfaces, raised edges, cutout regions, or recessed areas that align with corresponding features on the main body 116 to facilitate alignment and secure attachment during assembly of the monitoring device 104.

By way of example, as illustrated in the profile view 404 the overpatch 118 includes a recessed region 420. The recessed region 420, for instance, is dimensioned to accommodate components of the main body 116 and at least partially surround the main body 116. A top surface of the recessed region 420 is depicted to include the overpatch mating components 418 of the overpatch connection interface 416, such that the overpatch mating components 418 can contact the body mating components 322 disposed on the top surface 316 of the main body 116. In some examples, the overpatch 118 may include one or more mechanical interface components (e.g., protrusions, tabs, alignment pins, adhesives, snap-fit features, interlocking elements, etc.) that correspond to complementary mechanical interface components on the main body 116 such as to provide additional securing mechanisms in addition or alternative to the electrical connection established through the connection interfaces.

Further illustrated is an adhesive layer 422 that may be positioned on a skin-facing side of the overpatch 118. The adhesive layer 422, for instance, is configured to adhere to the skin surface of the user around a perimeter of the main body. The adhesive layer 422 can include various materials, such as acrylic adhesives, silicone-based adhesive materials, or other biocompatible adhesives to provide secure attachment to the skin surface during wear periods.

Accordingly, the overpatch 118 may have various components, geometries, and/or configurations to accommodate various monitoring applications and/or patient physiologies. In some embodiments, the overpatch 118 includes one or more rectangular shapes, circular shapes, extended wings or tabs, cutouts or openings, shapes contoured to body areas, rounded edges, reinforced areas, integrated strain relief features, varying thicknesses, alignment markers or guides, and so forth. In this way, the overpatch 118 may be configured for various monitoring scenarios, such as ECG-only versions, relatively larger versions with relatively more leads, versions for bioimpedance measurements, and so forth.

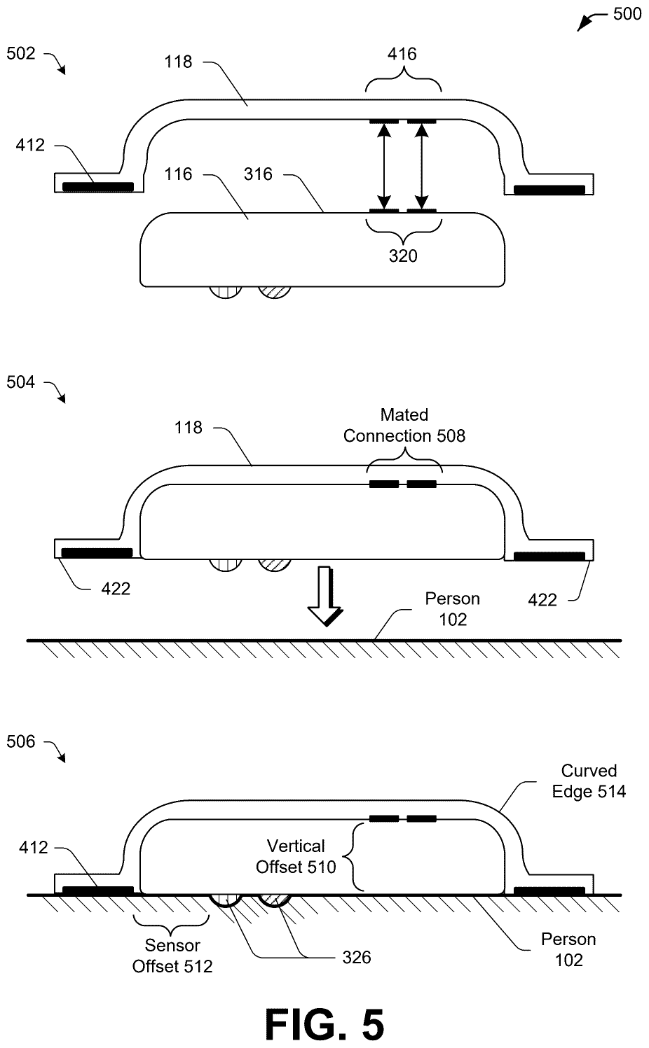

FIG. 5 depicts a non-limiting example 500 of attachment of an overpatch 118 to a main body 116 to attach a monitoring device 104 to a skin surface of a user in a first stage 502, second stage 504, and third stage 506. The example 500, for instance, depicts attachment of the main body 116 described with respect to FIG. 3 to the overpatch 118 described in FIG. 4.

In the first stage 502, the main body 116 and overpatch 118 are depicted as aligned with one another. The main body 116 includes the body connection interface 320 positioned on the top surface 316, while the overpatch 118 includes a corresponding overpatch connection interface 416 disposed along a top surface of the recessed region 420. As illustrated, the recessed region 420 is dimensioned to coincide with a geometry of the housing 308 of the main body 116 such as to partially or fully “cover” one or more portions of the main body 116.

The second stage 504 depicts the overpatch 118 and main body 116 connected via a mated connection 508 and disposed above a skin surface of a person 102, e.g., with an adhesive layer 422 facing the skin surface. The mated connection 508, for instance, includes the overpatch connection interface 416 connected to the body connection interface 320. The mated connection 508 thus forms an electrical connection between the body connection interface 320 and the overpatch connection interface 416. In this way, the mated connection 508 can enable transmission of physiological data 324 collected by one or more sensors 412 of the overpatch 118 to the processing circuit 310 housed within the main body 116.

In one or more examples, the main body 116 and/or the overpatch 118 may include one or more mechanical interface components to facilitate attachment to one another. By way of example and not limitation, the main body 116 may include one or more slots (e.g., a circumferential recessed indentation) dimensioned to receive corresponding protrusions of the overpatch 118 as a mechanical interface. In an additional or alternative example, the overpatch 118 includes one or more protrusions dimensioned to contact the bottom surface 318 of the overpatch 118, such as to “snap” the main body 116 within the recessed region 420. In some examples, the overpatch 118 includes a portion of the flexible substrate 406 configured to at least partially enclose the overpatch 118 via contact with the bottom surface 318. Thus, the monitoring device 104 can include various mechanical interface components to ensure alignment between electrical connection points and maintain secure attachment during user movement or physical activity.

Further, while not depicted in the illustrated example 500, in some implementations the main body 116 may include one or more additional sections connected to the housing 308. For instance, the overpatch 118 is attachable to an additional section that includes additional hardware to support additional operations, such as an additional section that includes additional sensors. Thus, the overpatch 118 is configurable in various ways to accommodate for various use cases.

The third stage 506 depicts the assembly, e.g., the monitoring device 104 that includes the attached main body 116 and overpatch 118, in contact with the skin surface of the person 102. For instance, the adhesive layer 422 contacts and adheres to the skin surface around a perimeter of the main body 116. In one or more examples, the adhesive layer 422 can provide a downward force such that the sensors 412 of the overpatch 118 as well as the optical sensors 326 of the main body 116 are in sufficient contact with the skin surface, such as to collect various physiological data 324. The adhesive layer 422 is thus usable to secure the assembled monitoring device 104 to the skin surface while maintaining various positional relationships provided by a geometry and/or configuration of the housing 308.

For instance, the main body 116 is dimensioned to position the mated connection 508 at a vertical offset 510 from the skin surface, such as a vertical offset 510 substantially equivalent to a height of the main body 116. The vertical offset 510 of the mated connection 508 can reduce incidence of biological signal noise. For example, by elevating the mated connection 508 away from the skin surface, the vertical offset 510 can reduce noise due to perspiration and/or moisture that may accumulate at the skin surface. The vertical offset 510 can further provide advantages such as reduced signal interference from skin movement, reduced effects of environmental factors, improved signal-to-noise ratio, and so forth.

Additionally or alternatively, the main body 116 can be dimensioned to position the sensor 412 at an offset distance, e.g., a sensor offset 512, from the optical sensors 326 located on the bottom surface 318 of the main body 116. The sensor offset 512 is a distance that is configured to reduce noise between the sensor 412 and the optical sensors 326 such as to prevent interference during physiological data collection. For instance, spatial separation provided by the sensor offset 512 can reduce noise due to proximity between various sensor types (e.g., ECG electrodes) and optical sensors 326, e.g., PPG sensors.

The main body 116 is further depicted to include one or more curved edges 514 between the top surface 316 and one or more side surfaces of the main body 116. The curved edges 514 may have a curve radius dimensioned to accommodate stress parameters of the conductive traces 414 to reduce mechanical stress on the conductive traces 414 during wear. For instance, a curve radius of the curved edge 514 may be based on a material, size, style, etc. of a conductive trace 414. Accordingly, the curved edges 514 may provide a smooth transition to enable the conductive traces 414 to follow relatively gradual paths rather than relatively sharp angles, which can reduce a likelihood of trace damage or failure during device movement or flexing.

FIG. 6 illustrates a non-limiting example 600 in which a main body 116 is compatible with multiple overpatch configurations.

In the example 600, the main body 116 is compatible with a first overpatch 602 and a second overpatch 604. For instance, the main body 116 includes two different body connection interfaces 320 such as a first body connection interface 606 and a second body connection interface 608. The first body connection interface 606 is configured to mate with a first overpatch connection interface 610 of the first overpatch 602, while the second body connection interface 608 is configured to mate with a second overpatch connection interface 612 of the second overpatch 604.

In various embodiments, the first overpatch 602 and the second overpatch 604 may have various sensor configurations, electrode arrangements, and/or support variable monitoring capabilities. For instance, the first overpatch 602 is depicted to include two sensors 412 (which are illustrated as large white circles) while the second overpatch 604 is depicted to include four sensors 412. Accordingly, the first overpatch 602 and second overpatch 604 are usable to support different monitoring scenarios.

In an example in which the first overpatch 602 attaches to the main body 116, the first overpatch connection interface 610 connects with the first body connection interface 606. Responsive to the connection, the processing circuit 310 can enter a first operational state 614. In various examples, the first operational state 614 may activate a particular subset of sensors 412 and/or cause implementation of one or more processing algorithms configured for particular physiological measurement scenarios. In an example in which the second overpatch connection interface 612 connects with the second body connection interface 608, the processing circuit 310 can enter a second operational state 616 that differs from the first operational state 614.

For example, the processing circuit 310 causes sensor activation 618 and/or implements a processing configuration 620 based on which overpatch is connected to the main body 116. For instance, connection of the first overpatch connection interface 610 with the first body connection interface 606 activates a first set of sensors and a first processing algorithm. Connection of the second overpatch connection interface 612 with the second body connection interface 608 activates a second set of sensors and a second processing algorithm.

The processing configuration 620 determines how the processing circuit 310 processes and/or stores physiological data 324 based on which overpatch connects to the main body 116. In one or more embodiments, the processing configuration 620 may include adjustments to signal processing parameters, data collection rates, or analysis algorithms based on the connected overpatch type. The processing configuration 620 may also modify power management settings and/or communication protocols of the monitoring device 104.

This functionality is useful for a variety of monitoring scenarios. By way of example and not limitation, the first overpatch 602 may be configured for a cardiac monitoring application that utilizes reduced power consumption, relatively few sensors, and a simplified processing algorithm such as to extend battery life during extended wear periods. In contrast, the second overpatch 604 incorporates additional sensors positioned at strategic locations to capture detailed physiological parameters, such as to support advanced analytics (e.g., arrhythmia detection or respiratory pattern analysis) that utilize relatively greater computational resources to provide enhanced diagnostic capabilities. This allows healthcare providers to select an appropriate configuration based on particular monitoring demands, such as to balance computational efficiency with diagnostic detail as clinically indicated.

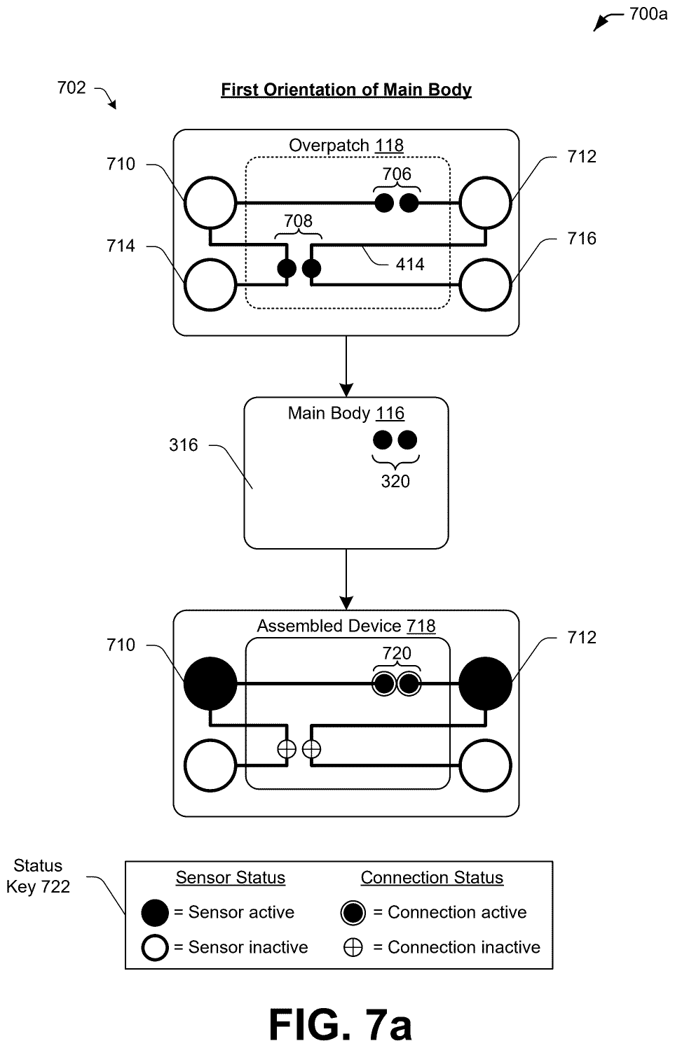

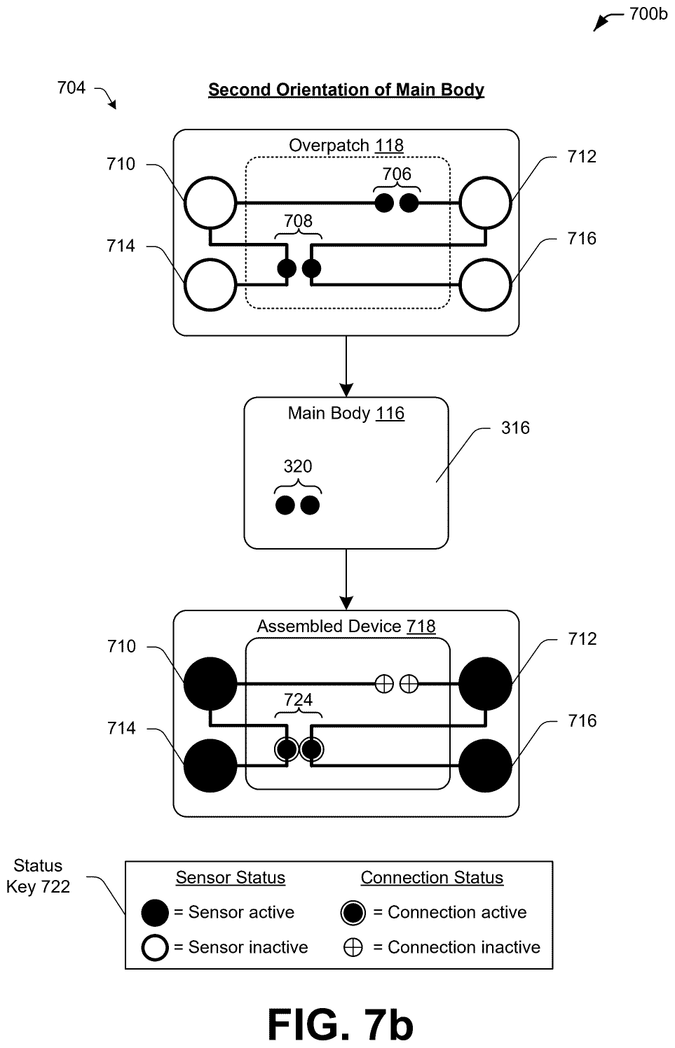

FIGS. 7a and 7b depict non-limiting examples 700a and 700b of orientation-dependent functionality of a wearable device assembly. For instance, the examples 700a, 700b demonstrate how different orientations of the main body 116 relative to the overpatch 118 may activate different sensor combinations and connection configurations in a first stage 702 and a second stage 704.

As shown in the first stage 702, an example overpatch 118 includes multiple connection interfaces and sensors. For instance, the overpatch 118 in this example includes a first overpatch connection interface 706 and a second overpatch connection interface 708 positioned at different locations on the overpatch 118. The overpatch 118 further includes a first sensor 710, a second sensor 712, a third sensor 714, and a fourth sensor 716. The first overpatch connection interface 706 is depicted as connected via conductive traces 414 to the first sensor 710 and the second sensor 712, while the second overpatch connection interface 708 is depicted as connected via different conductive traces 414 to the first sensor 710, second sensor 712, third sensor 714, and the fourth sensor 716.

In the first stage 702, the main body 116 is positioned in a first orientation relative to the overpatch 118. In the first orientation, the body connection interface 320 is disposed towards an “upper right” region of the top surface 316 as illustrated. The body connection interface 320 of the main body 116 aligns with the first overpatch connection interface 706 when the main body 116 and overpatch 118 are combined to form an assembled device 718. The connection between the body connection interface 320 and the first overpatch connection interface 706 creates a first mated connection 720 in accordance with the techniques described herein.

A status key 722 is provided that indicates an operational state of various sensors and connections within the assembled device 718. For instance, a filled large circle represents that a particular sensor 412 is active, while an unfilled large circle indicates that a particular sensor 412 is inactive. Similarly, a filled small circle disposed within an additional circle represents a particular connection interface component is active, while a small circle with an “x” through it indicates that a particular connection interface component is inactive.

In the example 700a, when the first mated connection 720 is formed between the body connection interface 320 and the first overpatch connection interface 706, the processing circuit 310 of the overpatch 118 causes the first sensor 710 and the second sensor 712 to be activated while maintaining the third sensor 714 and the fourth sensor 716 in an inactive state. The processing circuit 310 may further implement one or more operational states to collect, store, and/or process physiological data 324 using the first sensor 710 and the second sensor 712.

As shown in the second stage 704 in FIG. 7b, the main body 116 has been rotated approximately 180 degrees relative to the overpatch 118. For instance, the body connection interface 320 is shown disposed in a “lower left” portion of the top surface 316. In the second stage 704, the body connection interface 320 aligns with the second overpatch connection interface 708 rather than the first overpatch connection interface 706. The connection between the body connection interface 320 and the second overpatch connection interface 708 in the assembled device 718 forms a second mated connection 724.

The second mated connection 724 may cause a different sensor activation pattern and/or processing state relative to the first mated connection 720. For instance, upon establishment of the second mated connection 724, the processing circuit 310 activates the first sensor 710, the second sensor 712, the third sensor 714, and the fourth sensor 716 simultaneously.

Accordingly, the different orientations may correspond to different monitoring modes or measurement protocols. For instance, the first overpatch connection interface 706 and the second overpatch connection interface 708 may be electrically configured to activate different subsets of available sensors based on which interface establishes connection with the body connection interface 320. Additionally or alternatively, the assembled device 718 may include processing logic to identify which connection interface is active and adjust sensor activation and/or data processing algorithms accordingly. In some cases, the processing circuit 310 within the main body 116 may implement different signal processing parameters or measurement protocols based on whether the first mated connection 720 or the second mated connection 724 is established.

In this way, a single overpatch 118 can support multiple operational configurations based on an orientation of the main body 116 relative to the overpatch 118. Thus, the techniques described herein enhance device versatility relative to conventional approaches by obviating a demand for multiple specialized overpatches for different monitoring applications.

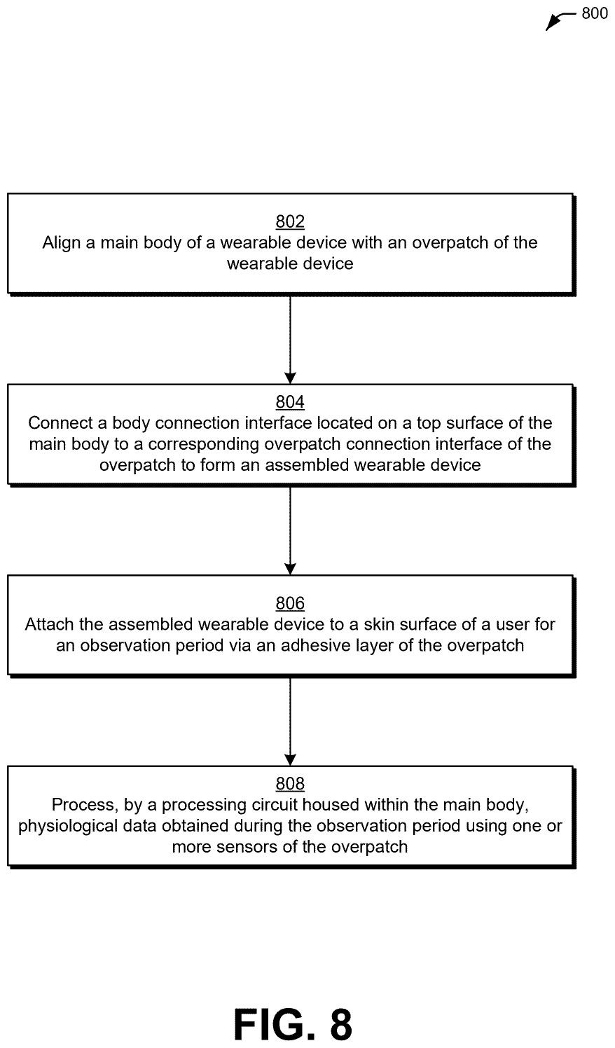

FIG. 8 depicts a flow diagram as a step-by-step procedure 800 in an example implementation that is performable to attach and operate a wearable device using a replaceable overpatch for physiological monitoring.

To begin in this example, a main body of a wearable device is aligned with an overpatch of the wearable device (block 802). The main body 116, for instance, may be positioned relative to the overpatch 118 such that a body connection interface 320 on a top surface 316 of the main body 116 corresponds with an overpatch connection interface 416 of the overpatch 118. In some examples, the overpatch 118 includes a recessed region 420 dimensioned to at least partially surround the main body 116 of the monitoring device 104. The alignment process thus can involve positioning the main body 116 within the recessed region 420 to facilitate proper connection between the respective connection interfaces.

A body connection interface located on a top surface of the main body is then connected to a corresponding overpatch connection interface of the overpatch to form an assembled wearable device (block 804). The body connection interface 320, for example, includes one or more pogo pins, mezzanine connectors, magnetic components, insertable zip connectors, contact pads, etc. that can mate with corresponding connectors of the overpatch connection interface 416. In various examples, the overpatch connection interface 416 includes one or more conductive pads or flexible circuit board terminals configured to mate with corresponding connectors of the body connection interface 320. The main body 116 and/or the overpatch 118 can include one or more mechanical interface components to mechanically secure the overpatch 118 to the main body 116.

The assembled wearable device is then attached to a skin surface of a user for an observation period via an adhesive layer of the overpatch (block 806). The overpatch 118, for instance, includes an adhesive layer 422 on a skin-facing side that is configured to adhere to the skin surface of the user. The overpatch 118 may be configured to prevent access to one or more service ports of the main body 116 during wear of the assembled wearable device, e.g., the monitoring device 104. In some cases, the flexible substrate 406 may be configured to prevent access to one or more service ports of the main body 116 when the overpatch 118 is attached to the monitoring device 104.

Physiological data obtained during the observation period is then processed by a processing circuit housed within the main body using one or more sensors of the overpatch (block 808). The processing circuit 310, for example, receives physiological data 324 from one or more sensors 412 embedded within the replaceable overpatch 118 through electrical connections formed between the body connection interface 320 and the overpatch connection interface 416. For instance, conductive traces 414 embedded within the overpatch 118 facilitate transmission of the physiological data 324 from the sensors 412 to the overpatch connection interface 416, which can communicate the physiological data 324 to the processing circuit 310 of the main body 116 for analysis and processing.

Upon termination of the observation period, the main body 116 may be detached from the overpatch 118 such as for reuse with subsequent monitoring sessions. In various examples, the overpatch 118 is a “single use” component that may be disposed of after the wear period. In this way, the main body 116 can be cleaned and prepared for attachment to an additional overpatch 118, thereby supporting cost-effective monitoring while maintaining hygiene standards between different users or monitoring periods.

It should be understood that many variations are possible based on the disclosure herein. Although features and elements are described above in particular combinations, each feature or element is usable alone without the other features and elements or in various combinations with or without other features and elements.

What is claimed is:

1. A wearable device attachable to a skin surface of a user to collect physiological data comprising:
a main body to house a processing circuit to process the physiological data, the main body including a bottom surface configured to face the skin surface during wear of the wearable device and a top surface substantially opposite the bottom surface that includes a body connection interface; and
an overpatch dimensioned to be placed over the main body to secure the main body to the skin surface that includes:
one or more sensors embedded within the overpatch to collect the physiological data; and
an overpatch connection interface dimensioned to mate with the body connection interface to form an electrical connection between the overpatch and the main body and communicate the physiological data to the processing circuit of the main body.

2. The wearable device of claim 1, wherein the one or more sensors include electrocardiogram (ECG) electrodes, and the overpatch includes conductive traces embedded within a flexible substrate that are electrically coupled to the ECG electrodes and to the overpatch connection interface.

3. The wearable device of claim 2, wherein the main body includes one or more curved edges between the top surface and one or more side surfaces of the main body, the one or more curved edges having a curve radius to accommodate stress parameters of the conductive traces to reduce mechanical stress on the conductive traces.

4. The wearable device of claim 1, wherein the overpatch includes a recessed region dimensioned to at least partially surround the main body and an adhesive layer on a skin-facing side of the overpatch configured to adhere to the skin surface of the user around a perimeter of the main body.

5. The wearable device of claim 1, wherein the body connection interface includes one or more pogo pins, mezzanine connectors, or insertable zip connectors, and the overpatch connection interface includes corresponding connectors configured to mate with the body connection interface.

6. The wearable device of claim 1, wherein the main body is dimensioned to position a mated connection between the body connection interface and the overpatch connection interface at a vertical offset from the skin surface at a height of the main body to reduce incidence of signal noise due to perspiration.

7. The wearable device of claim 1, wherein the bottom surface of the main body further includes one or more optical sensors to contact the skin surface to collect optical data and the overpatch is dimensioned to position the one or more sensors at an offset distance from the one or more optical sensors, the offset distance configured to reduce noise between the one or more sensors and the one or more optical sensors.

8. The wearable device of claim 1, wherein the overpatch is configured to prevent access to one or more service ports of the main body during wear of the wearable device.

9. The wearable device of claim 1, wherein the main body further includes one or more mechanical interface components, and the overpatch includes one or more corresponding mechanical interface components configured to engage with the one or more mechanical interface components of the main body to further secure the overpatch to the main body.

10. A replaceable overpatch to attach a main body of a wearable monitoring device to a user, the replaceable overpatch comprising:
a flexible substrate dimensioned to be placed over the main body of the wearable monitoring device, the flexible substrate including a sensing region to contact a skin surface of the user and a coupling region to attach the replaceable overpatch to the main body;
one or more sensors embedded within the sensing region to collect physiological data from the user;
one or more conductive traces embedded within the flexible substrate electrically coupled to the one or more sensors; and
an overpatch connection interface electrically coupled to the one or more conductive traces configured to mate with a corresponding body connection interface of the main body to enable transmission of the physiological data to a processing circuit of the main body.

11. The replaceable overpatch of claim 10, wherein the one or more sensors include electrocardiogram (ECG) electrodes embedded within the flexible substrate.

12. The replaceable overpatch of claim 10, wherein the sensing region includes an adhesive layer on a skin-facing side of the flexible substrate configured to adhere to the skin surface of the user and the coupling region includes a recessed region dimensioned to at least partially surround the main body of the wearable monitoring device.

13. The replaceable overpatch of claim 10, wherein the flexible substrate is configured to prevent access to one or more service ports of the main body when the replaceable overpatch is attached to the wearable monitoring device.

14. The replaceable overpatch of claim 10, further comprising one or more mechanical interface components configured to engage with corresponding mechanical interface components on the main body to secure the replaceable overpatch to the main body.

15. The replaceable overpatch of claim 10, wherein the overpatch connection interface includes one or more conductive pads or flexible circuit board terminals configured to mate with corresponding connectors of the body connection interface.

16. The replaceable overpatch of claim 10, further comprising an additional overpatch connection interface, and wherein connection of the overpatch connection interface with the body connection interface activates a first set of sensors and a first processing algorithm and connection of the additional overpatch connection interface with the body connection interface activates a second set of sensors and a second processing algorithm.

17. The replaceable overpatch of claim 10, wherein the flexible substrate includes at least one of a protective layer, a moisture-wicking layer, a conductive layer, an insulating layer, a breathable layer, a stretchable layer, or an antimicrobial layer.

18. A main body of a wearable device attachable to a skin surface of a user by a replaceable overpatch to collect physiological data from the user, the main body comprising:
a housing that includes a processing circuit to process the physiological data, a bottom surface configured to face the skin surface during wear of the wearable device, and a top surface substantially opposite the bottom surface; and
a body connection interface disposed on the top surface of the housing configured to form an electrical connection with a corresponding overpatch connection interface of the replaceable overpatch to receive the physiological data from one or more sensors of the replaceable overpatch.

19. The main body of claim 18, wherein the housing is dimensioned to position a mated connection between the body connection interface and the overpatch connection interface at a vertical offset from the skin surface at a height of the housing to reduce incidence of signal noise due to perspiration.

20. The main body of claim 18, further comprising an additional body connection interface, and wherein responsive to connection of the body connection interface with the overpatch connection interface the processing circuit activates a first set of sensors of the replaceable overpatch and a first processing algorithm and responsive to connection of the additional body connection interface with the overpatch connection interface the processing circuit activates a second set of sensors of the replaceable overpatch and a second processing algorithm.
